(19) United States
(12) Patent Application Publication
(10) Pub. No.: US 2011/0166514 A1
(43) Pub. Date: Jul. 7, 2011

(54) AUTOMATED SYSTEM FOR THE CONTROLLED DEPLOYMENT OF NESTED CANNULA

(75) Inventors: Karen I. Trovato, Putnam Valley, NY (US); Nicholas D. Marchuk, Evanston, IL (US)

(73) Assignee: KONINKLIJKE PHILIPS ELECTRONICS N.V., Eindhoven (NL)

(21) Appl. No.: 13/063,761

(22) PCT Filed: Sep. 1, 2009
(86) PCT No.: PCT/IB2009/053810

§ 371 Date: Mar. 14, 2011

Related U.S. Application Data
(60) Provisional application No. 61/097,233, filed on Sep. 16, 2008.

(51) Int. Cl. A61M 25/01

(52) U.S. Cl. 604/95.01

(57) ABSTRACT

A cannula control device (70) employs a platform (80) and one or more cannula control units (40). Each cannula control unit (40) includes a cannula (30), a rotation motor assembly (50) mechanically connected to the cannula (30) for rotating the cannula (30) to a specific rotational orientation relative to a calibration orientation associated with cannula control unit (40) and/or the platform (80), and a translation motor assembly (60) mechanically connected to the platform (80) for translating the cannula control unit (40) to a specific translational position relative to a calibration position associated with the cannula control unit (40) and/or the platform (80). The cannula(s) (30) of device (70) are capable of precisely reaching numerous target locations, particularly within an anatomical region of a same body or different bodies.

The present invention relates to a precise deployment and control of nested cannula of a nested cannula that enables the nested cannula to reach multiple locations within any anatomical region of a patient.

The use of minimally invasive procedures has grown in recent years due to their ability to allow for diagnosis or surgical treatment without the trauma typically resulting from open surgery. Minimally invasive surgical procedures can also allow for safe access to anatomical regions that were previously unreachable.

Typical tools utilized in minimally invasive surgical procedures can include rigid laparoscopic devices, robotic devices, or scopes that utilize marionette-like strings for control. Each of these devices imposes certain limitations and has inherent drawbacks. For instance, rigid laparoscopic devices can require open space for maneuvering both inside and outside the body. This space requirement can preclude the use of rigid laparoscopic devices in many types of procedures.

Robotic devices are unable to reach far into the human body since they rely on motors to control each joint angle. Motors are often large compared to the small anatomical spaces of the body. The number of robotic joints limits the complexity of the environment through which the robot can reach. Robots are often six degrees of freedom so that they can reach a fixed point in freespace at a particular orientation. The addition of anatomical obstacles effectively reduces the remaining active degrees of freedom. Additional motors to increase dexterity, also add weight and size. For example, robotic devices having seven degrees of freedom are often heavy and frequently hard to control smoothly.

Scopes that are controlled by marionette-like strings, such as bronchoscopes and endoscopes, rely on the marionette strings to control the distal part of the scope. Although thinner than a robotic device, control of only one arc at the distal end of the scope is also a significant limitation. Further, the use of marionette-like strings requires an additional increase in device radius.

Nested cannulas overcome these limitations by building the intended motion into the construction of a nested cannula so that motors and wires are unnecessary, and yet these small, thin devices are able to reach far into the human anatomy. Specifically, nested cannulas are typically made from several concentric, pre-curved, polymer or super elastic tubes that are configured in a specific way to reach a target, while avoiding anatomical “obstacles”. Each tube can telescope in and out of the others, and can also be spun. Interaction and manipulation of the tubes can be utilized by the physician for positioning the distal end of the tubes in the desired position.

The present invention provides a novel and unique motor control of nested cannula that facilitates a sequential motion, a simultaneous motion or a combination thereof of the nested cannula based on an independent rotation and translation of each cannula that expands the reach and re-use of the nested cannula.

One form of the present invention is a cannula control device employing a platform and at least one, but typically two or more cannula control units. Each cannula control unit includes a cannula, a rotation motor assembly mechanically connected to the cannula for rotating the cannula to a specific rotational orientation relative to a calibration orientation associated with the cannula control unit and/or the platform, and a translation motor assembly mechanically connected to the platform for translating the cannula control unit to a specific translational position relative to a calibration position associated with the cannula control unit and/or the platform.

A second form of the present invention is a cannula control system employing the cannula control device as described in the previous paragraph and one or more motor controllers in electrical communication with the cannula control device for selectively applying one or more motor activation signals to the cannula control device. Each motor activation signal is indicative of a planned deployment of the cannula(s), particularly a planned deployment of the cannula(s) within an anatomical region of a body (human or animal).

The foregoing form and other forms of the present invention as well as various features and advantages of the present invention will become further apparent from the following detailed description of various embodiments of the present invention read in conjunction with the accompanying drawings. The detailed description and drawings are merely illustrative of the present invention rather than limiting, the scope of the present invention being defined by the appended claims and equivalents thereof.

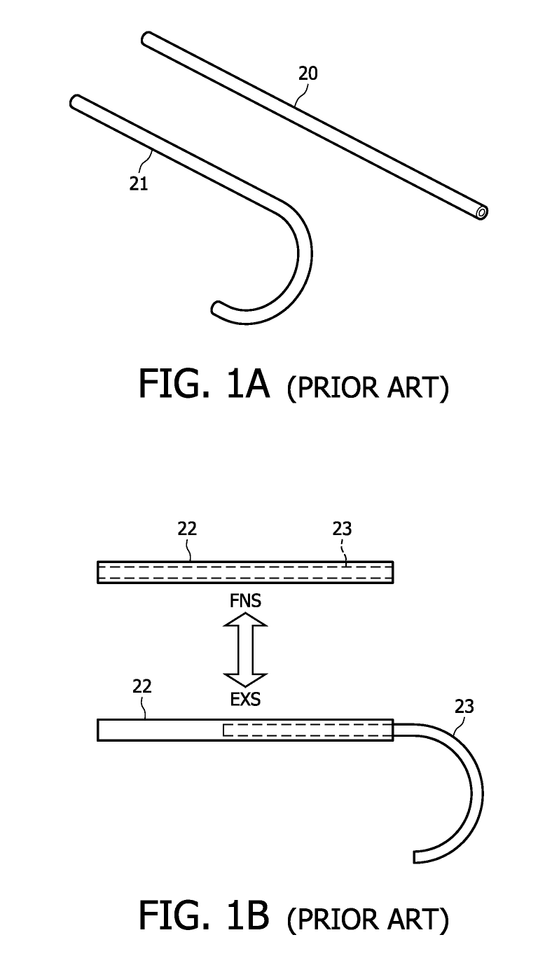

FIGS. 1A and 1B illustrate a pair of cannula as known in the art.

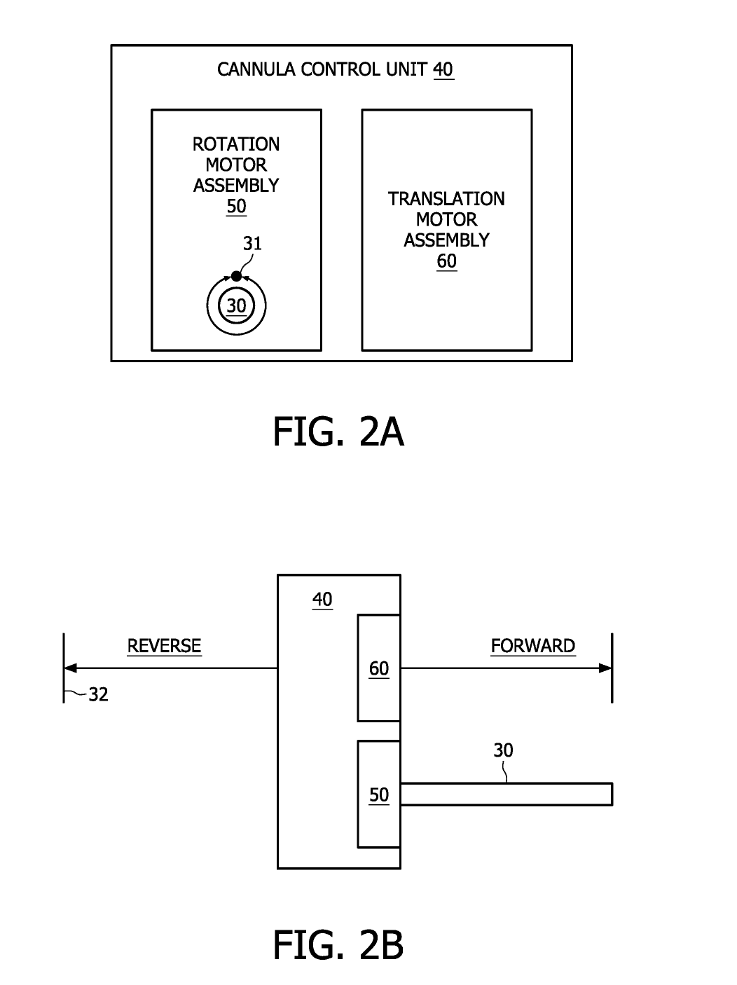

FIGS. 2A and 2B illustrate a front view and a side view, respectively, of a block diagram of an exemplary embodiment of a cannula control unit in accordance with the present invention.

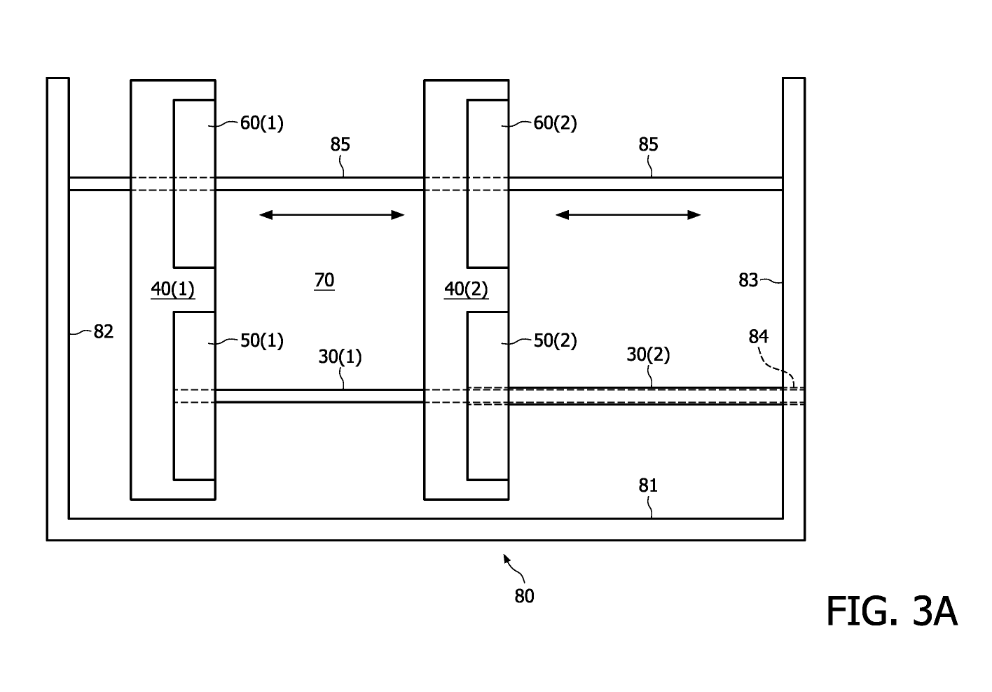

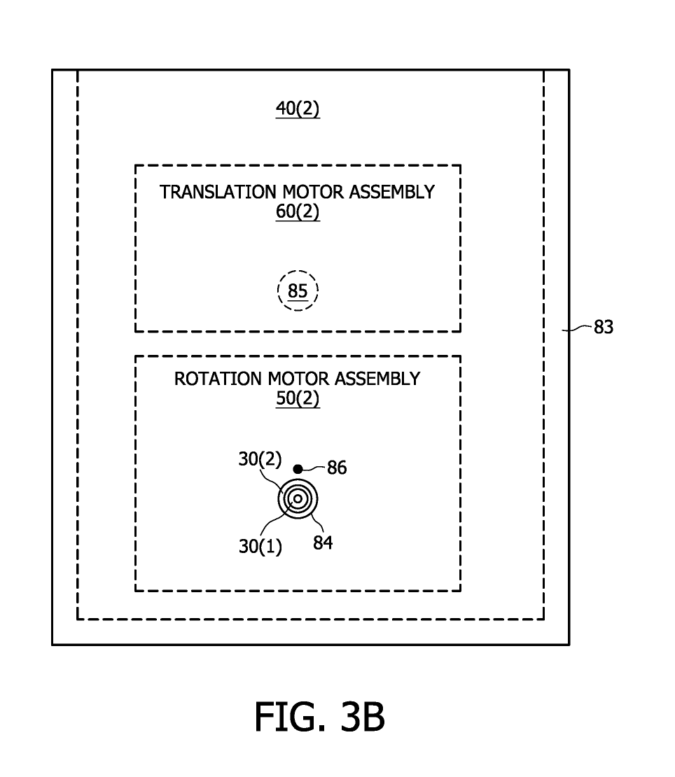

FIGS. 3A and 3B illustrate a side view and a front view, respectively, of an exemplary embodiment of a cannula control device in accordance with the present invention.

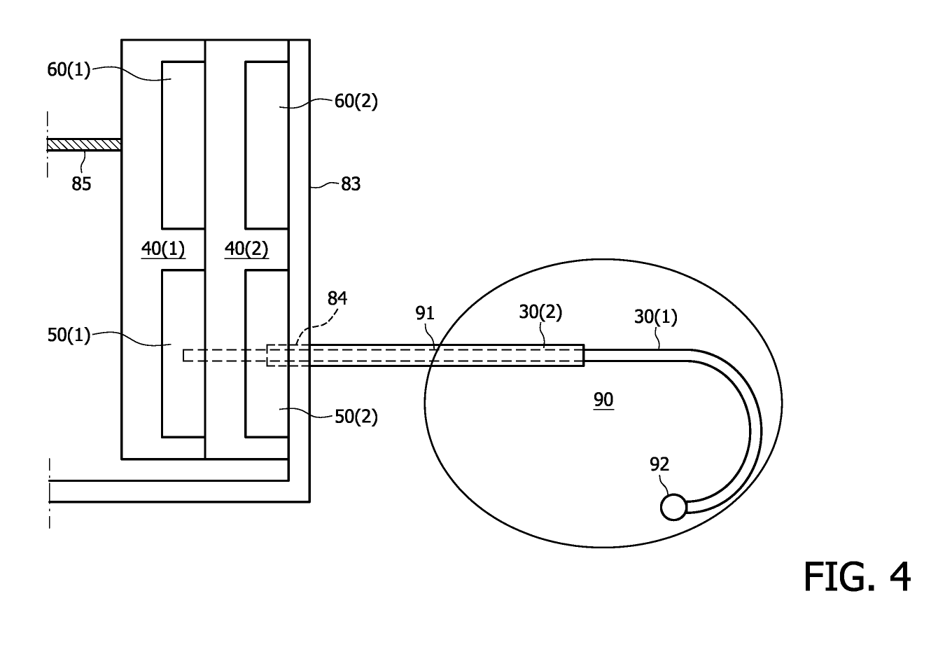

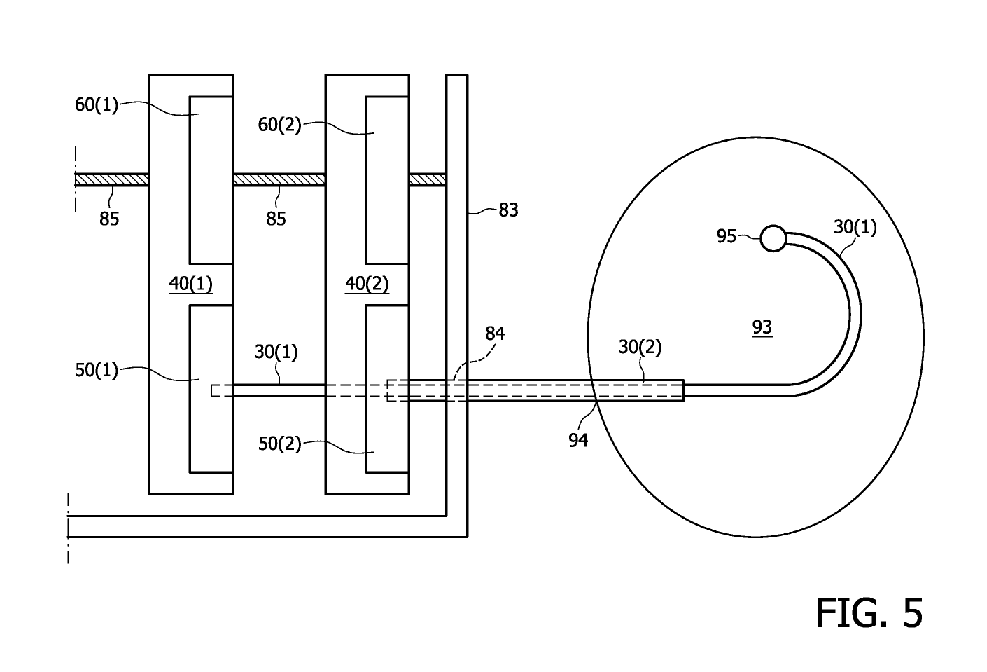

FIGS. 4 and 5 illustrate two (2) operational views of the cannula control device illustrated in FIGS. 3A and 3B.

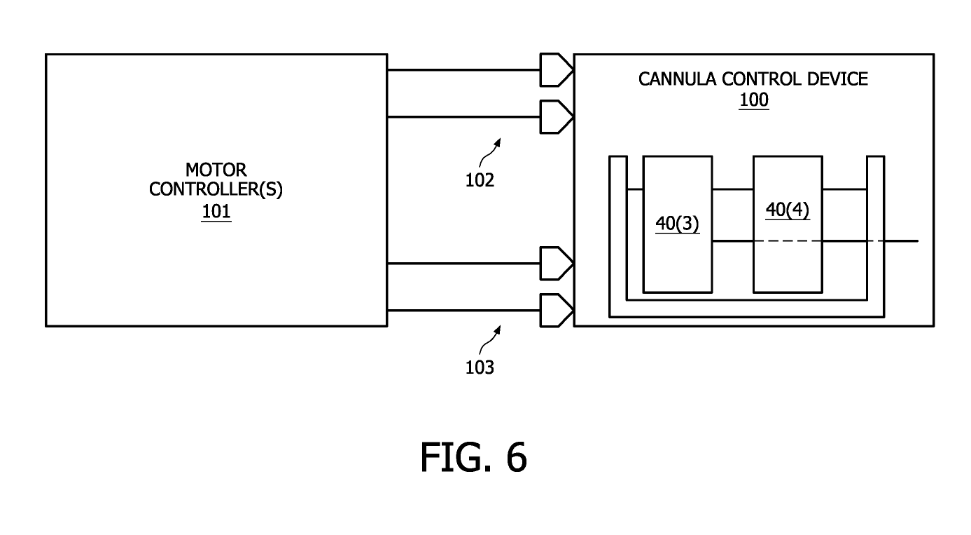

FIG. 6 illustrates an exemplary embodiment of a cannula control system in accordance with the present invention.

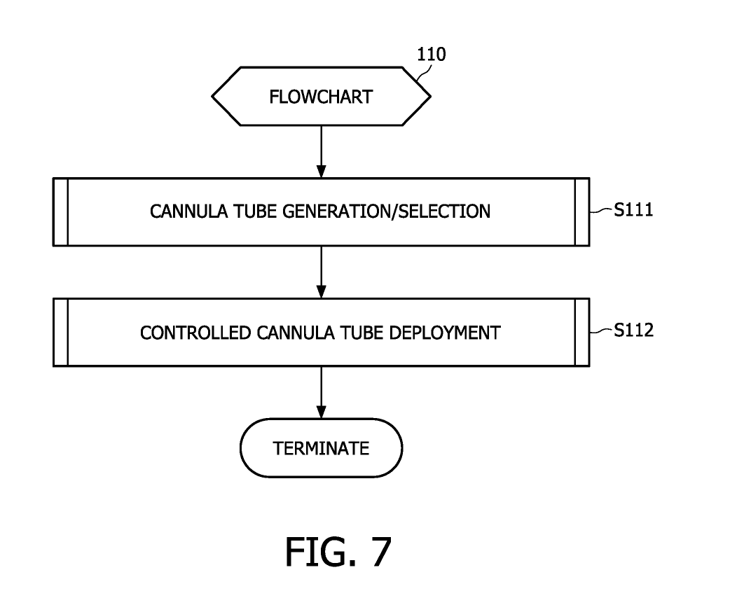

FIG. 7 illustrates a flowchart representative of an exemplary cannula control method in accordance with the present invention.

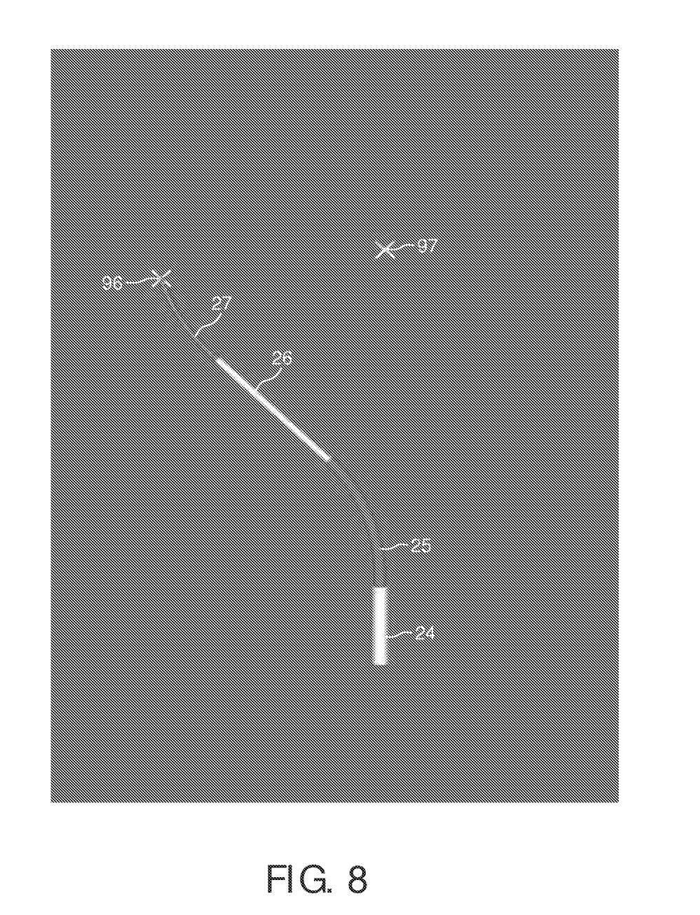

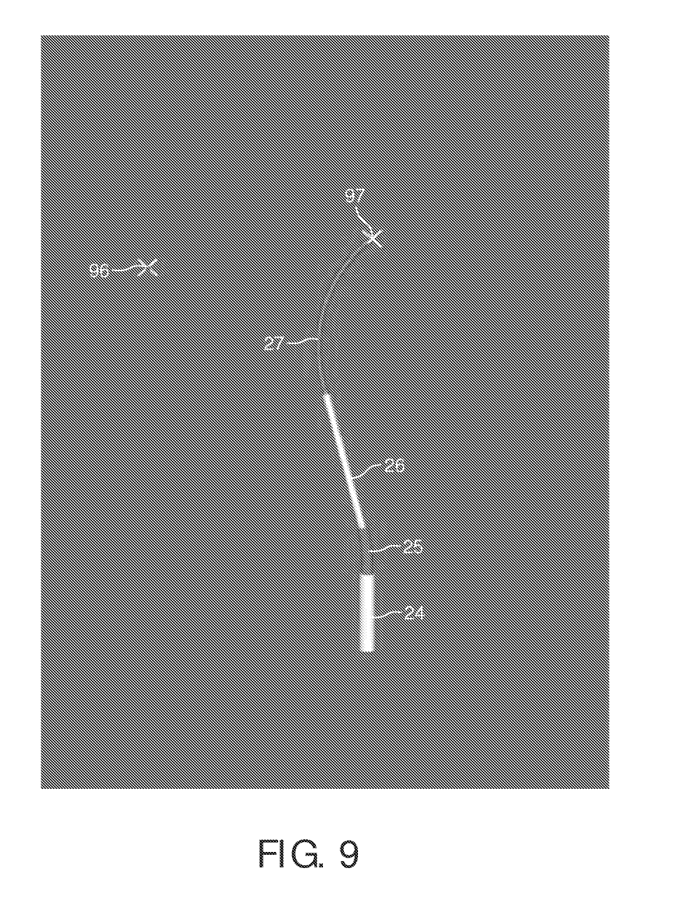

FIGS. 8 and 9 illustrate a re-use of a predefined set of cannula in accordance with the present invention.

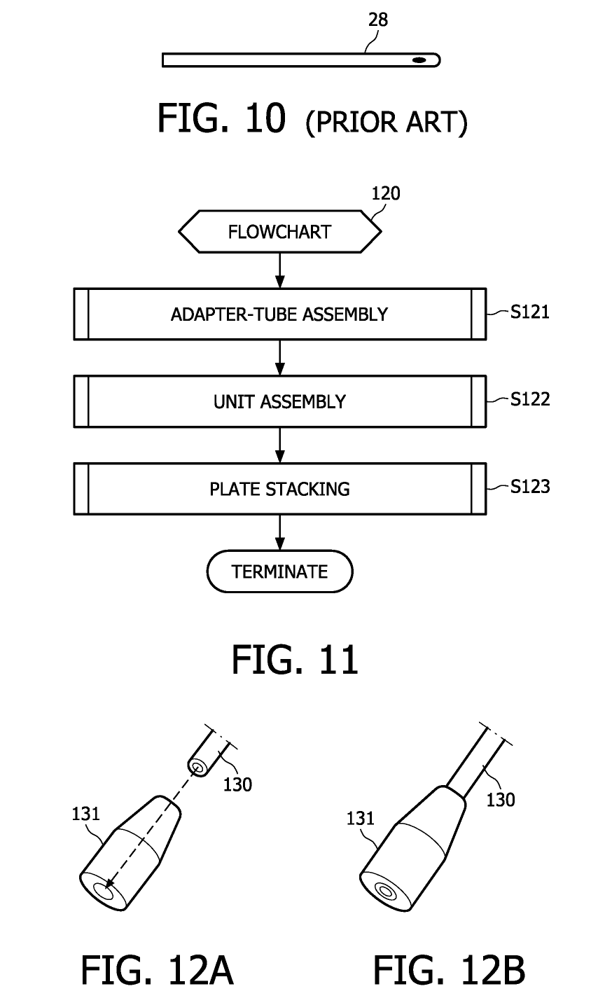

FIG. 10 illustrates a suction catheter as known in the art.

FIG. 11 illustrates a flowchart representative of an exemplary cannula control device construction method in accordance with the present invention.

FIGS. 12A-18 illustrate various stages of construction of a cannula control device in accordance with the flowchart shown in FIG. 11.

The present invention is directed to a controlled deployment of any type of cannula, such as, for example, a straight cannula 20 as shown in FIG. 1, a curved cannula 21 as shown in FIG. 1, or a helix cannula (not shown). As will be further explained herein, a controlled deployment of a single cannula in accordance with the present invention involves a rotation of the cannula to a specific rotational orientation relative to a calibration orientation. Alternatively or concurrently, the controlled deployment of a single cannula in accordance with the present invention involves a translation of the cannula to a specific translational position relative to a calibration position.

The present invention is further directed to a controlled deployment of any arrangement of nested cannulas, such as, for example, nested cannula 22 and 23 as shown in FIG. 2 whereby cannula 23 is rotatable within and movable between a fully nested state (“FNS”) and one of many extended states (“EXS”). As will be further explained herein, a controlled deployment of the nested cannula in accordance with the present invention involves a rotation of each cannula to a specific rotational orientation relative to a calibration orientation. Alternatively or concurrently, the controlled deployment of the nested cannula in accordance with the present invention involves a translation of each cannula to a specific translational position relative to a calibration position.

In practice, the configuration and dimensions of each cannula will be dependent upon the corresponding cannula procedure. Thus, the present invention does not impose any restrictions or any limitations on the configuration and dimensions of each cannula beyond any restriction or any limitation imposed by the corresponding cannula procedure.

Also, in practice, the cannula can made from various materials or combinations of materials including, but not limited to, a shape memory alloy (e.g., Nitinol), and/or a shape memory polymer (e.g., commercially available microtubes from Memry Inc, of Bethel, Conn. and MnemoScience GmbH of Aachen, Germany)) Polymers in general are a cost a cost-effective choice.

The premise of the present invention in controlling the deployment of a single cannula or nested cannula is the utilization of an X number of cannula control units 40 shown in FIG. 2A, where X≧1. Each cannula control unit 40 employs a rotation motor assembly 50 and a translation motor assembly 60.

For purposes of the present invention, a “rotation motor assembly” is broadly defined herein as any independent structural arrangement of a motor in conjunction with gear(s), screw(s), belt(s), sprocket(s), encoder(s), sensor(s) and/or other suitable electromechanical components for rotating cannula 30 to a specific rotational orientation relative to a calibration orientation, such as, for example, a rotation of cannula 30 to a specific rotational orientation relative to a calibration orientation 31 as shown in FIG. 2A. Alternatively or concurrently for purposes of the present invention, a “rotation motor assembly” is broadly defined herein as any independent structural arrangement of gear(s), screw(s), belt(s), sprocket(s), encoder(s), sensor(s) and/or other suitable electromechanical components mechanically connected to an external motor for rotating cannula 30 to a specific rotational orientation relative to a calibration orientation. As previously described herein, the configuration and the dimensions of cannula 30 will be dependent upon the corresponding cannula procedure.

Also, for purposes of the present invention, a “translation motor assembly” is broadly defined herein as any independent structural arrangement of a motor in conjunction with gear(s), screw(s), belt(s), sprocket(s), encoder(s), sensor(s) and/or other suitable electromechanical components for translating cannula control unit 40 in a forward motion or a reverse motion to a specific translational position relative to a calibration position, such as, for example, a linear translation of cannula control unit 40 relative to a calibration position 32 in a forward direction or a reverse direction as shown in FIG. 2B. Alternatively or concurrently for purposes of the present invention, a “translation motor assembly” is broadly defined herein as any independent arrangement of a motor in conjunction with gear(s), screw(s), belt(s), sprocket(s), encoder(s), sensor(s) and/or other suitable electromechanical components mechanically connected to an external motor for translating cannula control unit 40 in a forward direction or a reverse direction to a specific translational position relative to a calibration position.

To facilitate a further understanding of the inventive principles of cannula control unit 40, FIG. 3A illustrates an exemplary embodiment of a cannula control device 70 employing two (2) cannula control units 40(1) and 40(2) with cannula 30(1) of cannula control unit 40(1) being a curved cannula (e.g., curved cannula 21 shown in FIG. 1) and cannula 30(2) of cannula control unit 40(2) being a straight cannula (e.g., straight cannula 20 shown in FIG. 1).

Cannula control device 70 further employs a platform 80 having a base 81 and two (2) opposing walls 82 and 83 upwardly extending from base 81 to support a rail 85 along a length of base 81. Rail 85 extends through and is mechanically connected to each translation motor assembly 60 of the cannula control units 40 by any means that facilitates a forward or reverse direction of each cannula control unit 40 in an independent manner, a simultaneous manner or a combination thereof. In one embodiment, each translation motor assembly 60 has an independent internal motor for translating its respective cannula control unit 40 along rail 85 in a forward direction or a reverse direction to a specific translational position relative to a calibration position associated with cannula control unit 40 (e.g., an encoded baseline position of cannula control unit 40 along rail 85). Alternatively or concurrently, in another embodiment, a motor external to each translation motor assembly 60 rotates and/or translates rail 85 to thereby simultaneously translate the cannula control units 40 along rail 85 in a forward direction or a reverse direction to specific translational positions relative to a calibration position associated with cannula control unit 40 (e.g., an encoded baseline position relative to base 81 established by a servo motor).

A proximal end of each cannula 30 is mechanically connected by any means to the rotation motor assembly 50 of a respective cannula control unit 40 with the distal ends of cannula 30 being nested in a manner that facilitates the controlled deployment of the cannula 30 through a cannula channel 84 of front wall 83. In one embodiment, each rotation motor assembly 50 has an independent internal motor for rotating respective cannula control unit 40 to a specific orientation position relative to a calibration position associated with the respective cannula control unit 40 (e.g., an encoded baseline orientation of cannula control unit 40 along rail 85) and/or platform 80 (e.g., a baseline orientation 86 relative to cannula channel 84 as shown in FIG. 3B). Alternatively or concurrently, in another embodiment, a motor external to the cannula control units 40 independently imparts mechanical energy to each rotation motor assembly 50 to thereby rotate respective cannula control unit 40 to a specific orientation position relative to a calibration position associated with the respective cannula control unit 40 and/or platform 80.

In operation, FIG. 3A illustrates a fully nested state of cannula control device 70. In this nested state, cannula 30(1) has been pre-calibrated to a specific rotational orientation relative to a calibration orientation 85 associated with cannula channel 84.

FIG. 4 illustrates a fully extended state of the cannula control device shown in FIG. 3A within an anatomical region of a body 90. In this fully extended state, each translation motor assembly 60 has been activated to extend cannula 30 through cannula channel 84 and an entry point 91 of body 90 to specific positions within the anatomical region of body 90 to reach a target 92. As shown, cannula 30(2) maintains its straight configuration upon being extended from cannula channel 84, and cannula 30(1) resumes its arc configuration at a 180° rotational orientation relative to calibration orientation 86 (FIG. 3B) upon being extended from cannula 30(2).

FIG. 5 illustrates a partially extended state of the cannula control device shown in FIG. 3A within an anatomical region of a body 93. In this partially extended state, each translation motor assembly 60 is activated to translate cannula 30 through cannula channel 84 and an entry point 94 of body 93 to specific positions within the anatomical region of body 93 to reach a target 95. As shown, cannula 30(2) maintains its straight configuration upon being extended from cannula channel 84, and cannula 30(1) resumes its arc configuration at a 0° rotational orientation relative to calibration orientation 86 (FIG. 3B) upon being extended from cannula 30(2).

FIGS. 3-5 are simple illustrations of a cannula control device of the present invention that highlights a significant benefit of the present invention. Specifically, in view of the operational nature of motor assemblies 50 and 60, the distance cannula 30 are extended from cannula channel 84 as shown in FIG. 4 is greater than the distance cannula 30 are extended from cannula channel 84 as shown in FIG. 5, and the angular orientations of cannula 30(1) have a 180° differential as shown in FIGS. 4 and 5. This highlights the fact that a single set of cannula can be re-used from the same nesting state to reach numerous targets within the same body, and with proper sterilization, different targets within different bodies as shown in FIGS. 4 and 5.

To facilitate an even further understanding of the inventive principles of cannula control unit 40, FIG. 6 illustrates a cannula control system employing a cannula control device 100 having two (2) cannula control units 40(3) and 40(4) and one or more motor controllers 101. For purposes of the present invention, a “motor controller” is broadly defined herein as any device structurally configured for selectively applying motor activation signals (e.g., setpoints) to a cannula control device of the present invention in execution of a planned deployment of the cannula(s).

For example, motor controller(s) 101 is(are) shown in FIG. 6 as applying a signal set 102 of a rotation activation signal and a translation activation signal to cannula control device 100 for purposes of deploying the cannula of cannula control unit 40(3) in execution of a planned deployment of this particular cannula. In response to the rotation activation signal, the rotation motor assembly (not shown) of cannula control unit 40(3) rotates its cannula to a specific rotational orientation relative to a calibration orientation as indicated by the rotation activation signal. In the case where the cannula of cannula control unit 40(3) has a straight configuration, rotation activation signal may have a null value or be omitted from signal set 102. In response to the translation activation signal, the translation motor assembly (not shown) of cannula control unit 40(3) translates its cannula to a specific translational position relative to a calibration position as indicated by the translation activation signal.

Similarly, motor controller(s) 101 is(are) shown in FIG. 6 as applying a signal set 103 of a rotation activation signal and a translation activation signal to cannula control device 100 for purposes of deploying the cannula of cannula control unit 40(4) in execution of planned deployment of this particular cannula. In response to the rotation activation signal, the rotation motor assembly (not shown) of cannula control unit 40(4) rotates its cannula to a specific rotational orientation relative to a calibration orientation as indicated by the rotation activation signal. In the case where the cannula of cannula control unit 40(4) has a straight configuration, rotation activation signal may have a null value or be omitted from signal set 103. In response to the translation activation signal, the translation motor assembly (not shown) of cannula control unit 40(4) translates its cannula to a specific translational position relative to a calibration position as indicated by the translation activation signal.

Those having ordinary skill in the art will appreciate that the cannula may need to maintain their relative positions as the cannula are being translated within an anatomical region of a body, such as, for example, when the corresponding cannula procedure requires an insertion of a tool or the like that needs to maintain a position ahead of the cannula as the cannula are being translated within the anatomical region of the body. Therefore, alternatively, the rotation of the cannula of cannula control units 40(3) and 40(4) remain independent while the translation of the cannula are performed in a simultaneous manner. Specifically, signal set 102 can represent rotation activation signal(s) to rotate the cannula(s) to specific rotational orientation(s). By comparison, signal set 103 can represent a forward translation signal for concurrently translating the cannula in a forward direction and a reverse translation signal for concurrently translating the cannula in a reverse direction.

A description of a cannula control method of the present invention as represented by a flowchart 110 shown in FIG. 7 will now be described herein in the context of a cannula procedure involving a deployment of cannula within an anatomical region of a body. From these description of flowchart 110, those having ordinary skill in the art will appreciate how to apply the cannula control method of the present invention to other types of cannula procedures.

Specifically, a stage S111 of flowchart 110 encompasses a cannula generation scheme and a cannula selection scheme. In the generation scheme, stage S111 generally incorporates (a) a reading of a three dimensional image of the anatomical region of the body (e.g., CT, Ultrasound, PET, SPECT, MRI), (b) a generation of a series of arcs from a particular position and orientation in the three dimensional image, (c) a use of the generated series of arcs to calculate of a pathway through the body between an entry and target location using the generated series of arcs passing through the point, (d) a use of the generated series of arcs and the calculated pathway to generate one or more concentric telescoping tubes that are configured and dimensioned to reach the target location, and (e) a mechanical connection of each cannula to rotational motor assembly of a cannula control unit. In the selection scheme of the present invention, stage S111 generally incorporates (a) a reading of a three dimensional image of the anatomical region of the body, (b) a calculation of a pathway through the body between an entry and target location, and (c) a selection of one or more cannula control units having previously generated cannula(s) configured and dimensioned to reach the target location.

For example, the following Table 1 lists a configuration of four (4) nested cannula 24-27 for reaching a target location 96 as shown in FIG. 8 and for reaching a different target location 97 as show in FIG. 9:

TABLE 1

| | Calibrated | Extended Length | | Extended Length | |
|---|---|---|---|---|---|
| Tube Type | Orientation | (FIG. 19) | | (FIG. 20) | |
| Outer | N/A | 16 | mm | 16 | mm |
| Straight 24 | | | | | |
| Intermediate | Counterclockwise | 28.8 | mm | 10 | mm |
| 35 mm | 45 degrees | | | | |
| Curved 25 | | | | | |
| Intermediate | N/A | 30 | mm | 28 | mm |
| Straight 26 | | | | | |
| Inner | Clockwise + | 20.2 | mm | 33.6 | mm |
| 35 mm | 90 degrees | | | | |
| Curved 27 | | | | | |

The ‘Extended Length’ above describes the length that extends beyond the enclosing tube. Therefore the total length of the Intermediate Curved tube equals 16 mm plus 28.8 mm=44.8 mm, plus the length required to reach through a cannula guide channel (e.g., channel 84 shown in FIG. 3A). In this example, the change in lengths of the tubes enables the same set of tubes to reach from the target location 96 to target location 97 within the same body or a different body.

Irrespective of the scheme, those having ordinary skill in the art will appreciate that a straight cannula has one (1) degree of freedom that enables the tube to be advanced and retracted in accordance with it extendable length. By comparison, a curved cannula has two (2) degrees of freedom that enables the tube to be advanced and retracted in accordance with its extendable length, and rotated in accordance with its radius. A straight tube with a sensor or actuator set along the side, or carrying an end effector that has a specific orientation must similarly be considered to have 2 degrees of freedom since it has a unique orientation. Preferably, a curved cannula is only advanced the length corresponding to the curvature of the tube, for example the length of the arc of 180 degrees=π*radius.

Referring again to FIG. 7, a stage S112 of flowchart 110 encompasses a controlled deployment of the cannula, generated or selected, through the entry point to the target. Specifically, stage S112 generally incorporates (1) a selective application of the motor activation signals indicative of the planned path through the anatomical region of the body to the various motor assemblies of the cannula control device, (2) a registration of the cannula within an image space of anatomical region of the body as the cannula are being deployed in execution of the planned path, and (3) a real-time determination of an absolute translational position and rotational orientation (if applicable) of each deployed cannula within the image space via absolute encoders (e.g., potentiometers) to correct for any deviation from the planned path through the anatomical region of the body.

Referring to FIGS. 1-9, those having ordinary skill in the art will appreciate the various benefits of the present invention including, but not limited to, a precise controlled deployment of a single cannula or nested cannula in execution of a planned deployment of the cannula(s) for any type of cannula procedure. In particular, the cannula(s) are capable of precisely reaching numerous target locations within an anatomical region of a same body or different bodies (e.g., thoracic regions, abdominal regions, neurological regions, cardiac regions and vascular regions).

Furthermore, those having ordinary skill in the art will appreciate how to make and use a cannula control device of the present invention for any type of cannula procedure based on the general description of the invention principles of the present invention as illustrated of FIGS. 1-9. In particular, the use of nested cannula can be extended to allow for access to a targeted anatomical region by passing a tool or other device through the extended tubes. Alternatively, the inner-most tube itself can be a tool or include another device. For example, (1) the inner-most tube can be a suction catheter 28 as shown in FIG. 10 for laparoscopic procedures and the like, (2) the inner-most tube can have an imaging device at an end thereof so that when extended from the nested state the imaging device is positioned in the targeted anatomical region, (3) the inner-most tube can have a closed end, such as, for example, if it is a fiber optic line for transmitting light to and/or from the targeted anatomical region, and (4) the inner most tube can be substantially or completely solid as needed.

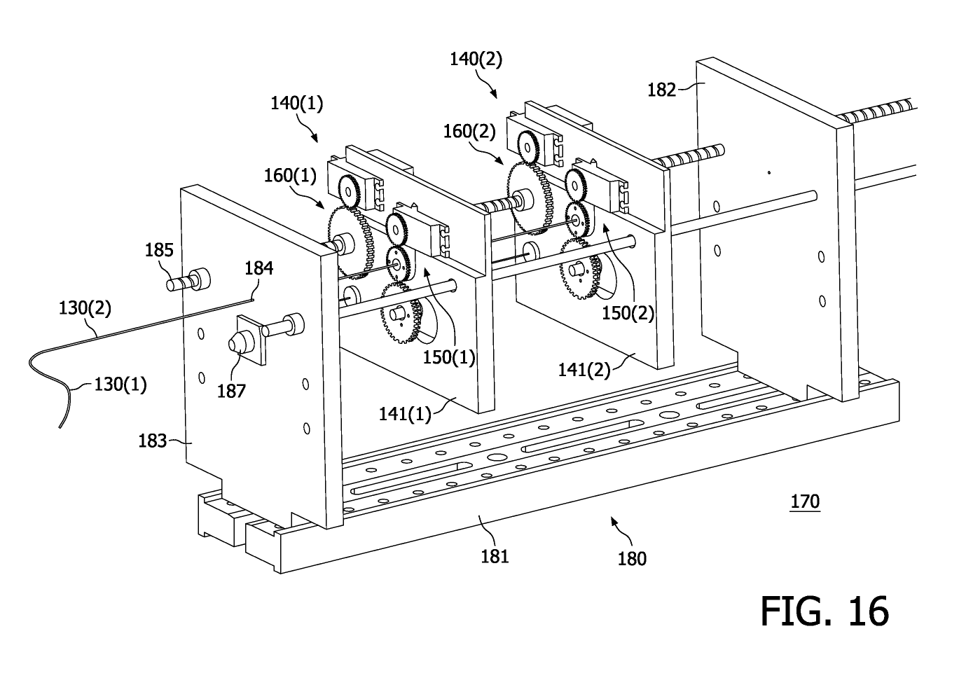

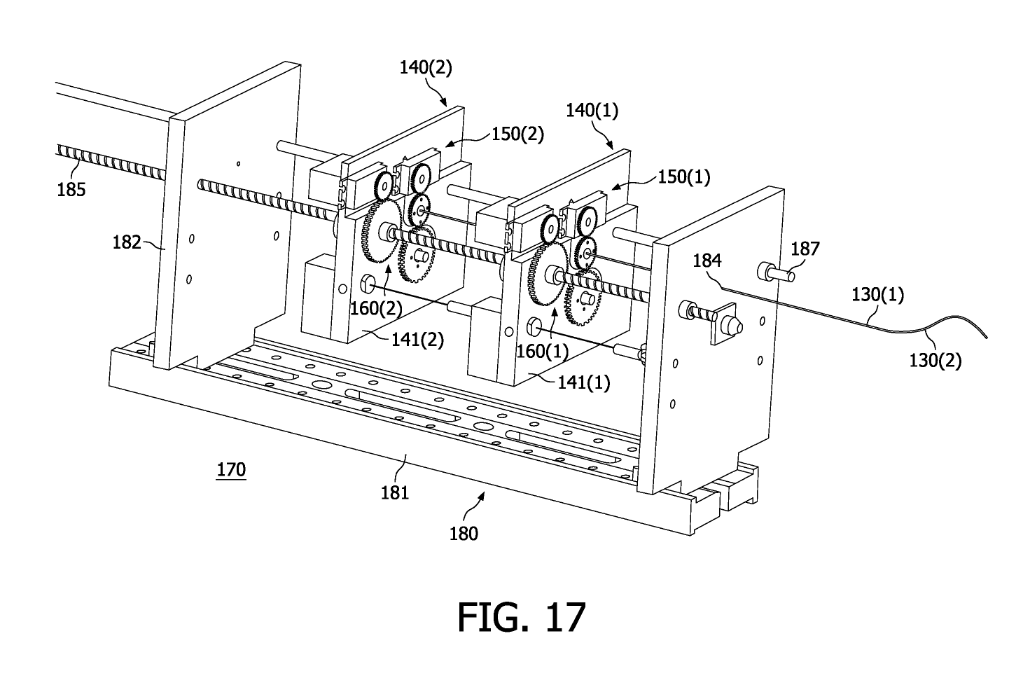

A detailed embodiment of a cannula control device 170 having two (2) cannula control units 140(1) and 140(2) in accordance with the present invention as shown in FIGS. 16 and 17 will now be provided herein in accordance with a flowchart 120 shown in FIG. 11.

Specifically, a stage S121 of flowchart 120 encompasses an adapter-cannula assembly for each generated cannula 130. In this embodiment, stage S121 involves a proximal end of a generated cannula 130 being friction fitted within a tub-hub adapter 131 as shown in FIG. 12A whereby the proximal end of cannula 130 is flush with a rear surface of adapter 131 as shown in FIG. 12B.

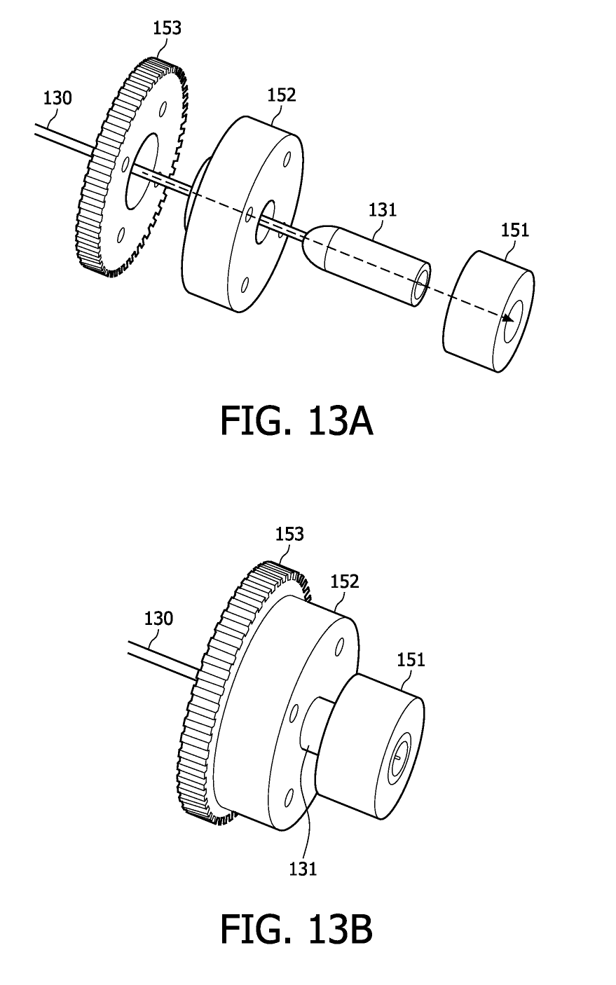

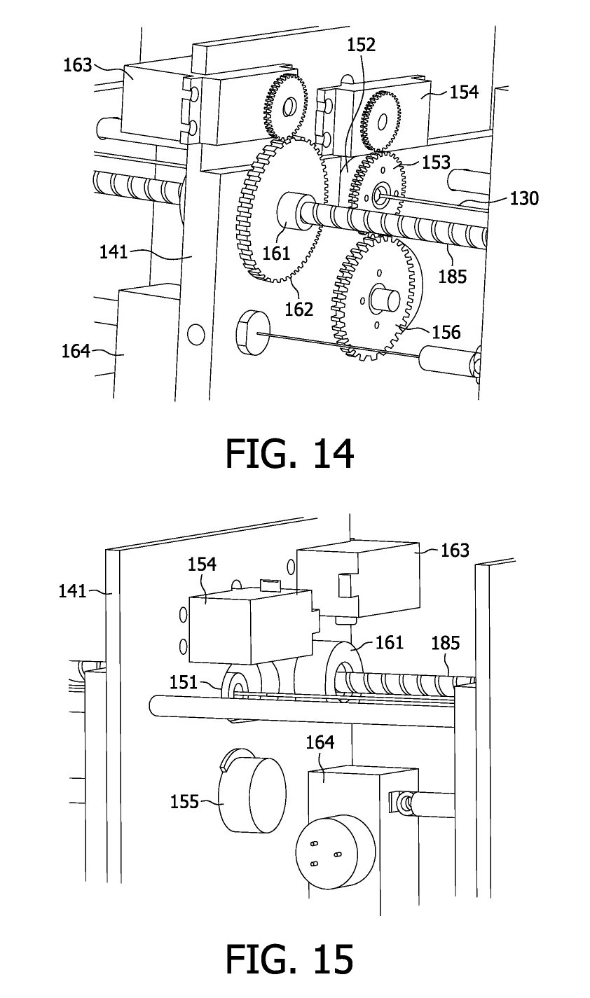

A stage S122 of flowchart 120 encompasses a unit assembly of each cannula control unit 140. In this embodiment, stage S122 involves adapter 131 being “dead ended” within a rotation motor assembly 150, such as, for example, adapter 131 being friction fitted within a calibration collar 151 of rotation motor assembly 150 as shown in FIG. 13A whereby the rear surface of adapter 131 is flush with a rear surface of calibration collar 151 as shown in FIG. 13B. Thereafter, stage 122 further involves the distal end of cannula 130 being sequentially fed through feed hole of a plate 141 as shown in FIGS. 14 and 15 and then fed through a hub 152 and a gear 153 of rotation motor assembly 150 as shown in FIGS. 12B-15 whereby cannula 130 and adapter 131 are locked down to plate 141 via a set screw (not shown).

In conjunction with locking cannula 130 and adapter 131 to plate 141, the remaining components of assembly 150 including a servo motor 154, a rotational encoder 155 and a gear 155 are assembled as shown in FIGS. 14 and 15. Furthermore, the components of a translation motor assembly 160 including a threaded adapter 161, a gear 162, a servo motor 163 and a linear encoder 164 as assembled as shown in FIGS. 14 and 15.

A stage S123 of flowchart 120 encompasses a plate stacking of the cannula control units 140 onto a platform 180 having a base 181 and opposing parallel walls 182 and 183 supporting a threaded rail 185 therebetween as shown in FIGS. 16 and 17. In this embodiment, stage 5123 involves a distal end of cannula 130(2) being inserted within the larger enclosing tube 130(1), and a distal end of tube 130(1) being output feed of platform 180 as shown in FIGS. 16 and 17. Stage S123 further involves a threaded rail 185 being threaded through translation motor assemblies 160(1) and 160(2) as shown in FIGS. 16 and 17, and a supplemental guide 187 being inserted through plates 141 as shown in FIGS. 16 and 17.

The result, as shown in FIGS. 16 and 17 is rotation motor assembly 150(1) and a translation motor assembly 160(1) being mechanically connected to plate 141(1) as and a rotation motor assembly 150(2) and a translation motor assembly 160(2) being secured to a plate 141(2) with a nesting of cannula 130(1) and 130(2).

In operation, a servo motor 154 of a rotation motor assembly 150 as shown in FIGS. 14-17 will receive a motor activation signal in execution of a planned deployment of the corresponding cannula 130 to rotate the cannula 130 as needed, and a servo motor 163 of a translation motor assembly 160 as shown in FIGS. 14-17 will receive a translation activation signal in execution of a planned deployment of the corresponding cannula 130 to translate the cannula 130 as needed. In the illustrated embodiment, the translation motor assemblies 160(1) and 160(2) are independently operated via distinct translation activation signals. Alternatively or concurrently, in view of threaded rail 185 being rotatable, a rotation of threaded rail 185 will simultaneously translate both cannula control units 140(1) and 140(2) in a forward direction or reverse direction. This requires base 181 to incorporate a servo motor (not shown) to actuate threaded rail 185. This embodiment is particularly useful for calibration of cannula 130(1) and 130(2).

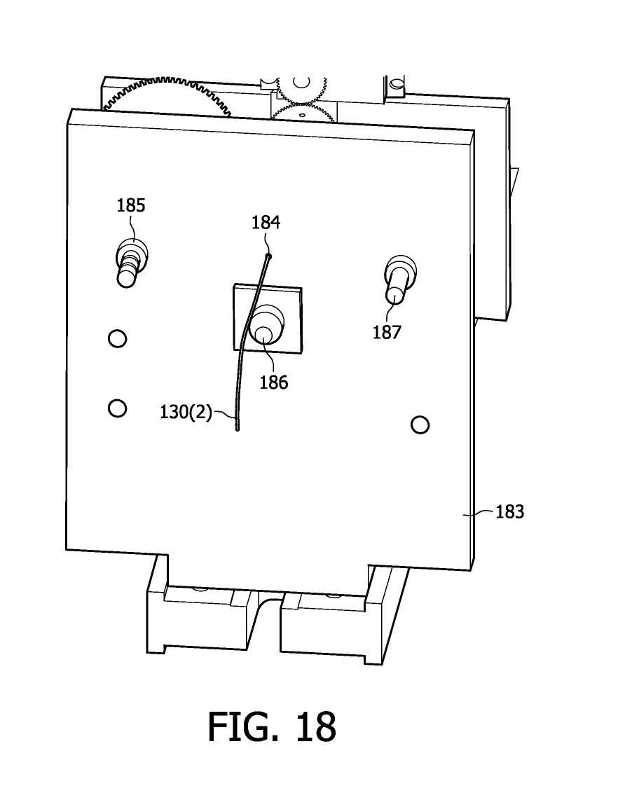

One design consideration is the fact that cannula 130(2) must be longer than the larger enclosing cannula 130(1), and cannula 130(2) contains an arc at the end that must calibrated to a specific rotational orientation One way to calibrate cannula 130(2) is to move the translatable plate 141 to a position whereby the surrounding tube 130(2) does not interfere with the natural arc shape of cannula 130(2) as shown in FIG. 18 where the arc of cannula 130(2) extends out cannula guide channel 184 of wall 183. A laser light 186 as shown in FIG. 18 can be mounted on wall 183 adjacent channel 184, or another calibrating structure can be used to define the aligned calibration orientation. In one embodiment, cannula 130(2) can be rotated by hand to the calibration orientation, and then the set-screws for the gear-hubs can be tightened with the orientation offset, if any, being stored programmatically. In a second embodiment, tube 130(2) can be tightened onto assembly 15092) and driven with the servo motor 157 to the calibration orientation 186 with the orientation offset, if any, being stored programmatically.

While various embodiments of the present invention have been illustrated and described, it will be understood by those skilled in the art that the methods and the system as described herein are illustrative, and various changes and modifications may be made and equivalents may be substituted for elements thereof without departing from the true scope of the present invention. In addition, many modifications may be made to adapt the teachings of the present invention to entity path planning without departing from its central scope. Therefore, it is intended that the present invention not be limited to the particular embodiments disclosed as the best mode contemplated for carrying out the present invention, but that the present invention include all embodiments falling within the scope of the appended claims.

1. A cannula control device (70), comprising:
a platform (80); and
at least one cannula control unit (40), wherein each cannula control unit (40) includes
a cannula (30),
a rotation motor assembly (50) mechanically connected to the cannula (30) for rotating the cannula (30) to a specific rotational orientation relative to a calibration orientation associated with at least one of the cannula control unit (40) and the platform (80), and
a translation motor assembly (60) mechanically connected to the platform (80) for translating the cannula control unit (40) to a specific translational position relative to a calibration position associated with at least one of the cannula control unit (40) and the platform (80).

2. The cannula control device (70) of claim 1,
wherein a first cannula control unit (40) further includes an adapter (131) friction fitted within a first rotation motor assembly (50); and
wherein a proximal end of the first cannula (30) is friction fitted within the adapter (131) to thereby mechanically connect the first cannula (30) to the first rotation motor assembly (50).

3. The cannula control device (70) of claim 2,
wherein the first cannula control unit (40) further includes a translatable plate (141); and
wherein a distal end of the first cannula (30) is feed through the translatable plate (141).

4. The cannula control device (70) of claim 1, wherein the platform (80) includes a rail (85) threaded through each translation motor assembly (60) to thereby mechanically connect each translation motor assembly (60) to the platform (80).

5. The cannula control device (70) of claim 1, wherein the platform (80) includes a front wall (83) having a cannula guide channel for facilitating an extension of at least one cannula (30) from the platform (80).

6. The cannula control device (70) of claim 1, wherein the platform (80) further includes a calibration mechanism (186) mounted on the front wall (83) for establishing the calibration orientation.

7. The cannula control device (70) of claim 1, wherein the at least one cannula (30) includes at least one of a straight cannula, a curved cannula and a helix cannula.

8. A cannula control system, comprising:
a cannula control device (70) including
a platform (80), and
at least one cannula control unit (40), wherein each cannula control unit (40) includes
a cannula (30),
a rotation motor assembly (50) mechanically connected to the cannula (30) for rotating the cannula (30) to a specific rotational orientation relative to a calibration orientation associated with at least one of the cannula control unit (40) and the platform (80), and
a translation motor assembly (60) mechanically connected to the platform (80) for translating the cannula control unit (40) to a specific translational position relative to a calibration position associated with at least one of the cannula control unit (40) and the platform (80); and
at least one motor controller in electrical communication with the cannula control device (70) for selectively applying motor activation signals to cannula control device (70), wherein the motor activation signals are indicative of a planned deployment of the at least one cannula (30).

9. The cannula control system of claim 8,
wherein a first cannula control unit (40) further includes an adapter (131) friction fitted within a first rotation motor assembly (50); and
wherein a proximal end of the first cannula (30) is friction fitted within the adapter (131) to thereby mechanically connect the first cannula (30) to the first rotation motor assembly (50).

10. The cannula control system of claim 8,
wherein the first cannula control unit (40) further includes a translatable plate (141); and
wherein a distal end of the first cannula (30) is feed through the translatable plate (141).

11. The cannula control system of claim 8, wherein the platform (80) includes a rail (85) threaded through each translation motor assembly (60) to thereby mechanically connect each translation motor assembly (60) to the platform (80).

12. The cannula control system of claim 8, wherein the platform (80) includes a front wall (83) having a cannula guide channel for facilitating an extension of at least one cannula (30) from the platform (80).

13. The cannula control system of claim 8, wherein the platform (80) further includes a calibration mechanism (186) mounted on the front wall (83) for establishing the calibration orientation.

14. The cannula control system of claim 8, wherein the at least one cannula (30) includes at least one of a straight cannula, a curved cannula and a helix cannula.

15. In a cannula control unit (40) including a cannula (30), a rotation motor assembly (50) mechanically connected to the cannula (30), and a translation motor assembly (60) mechanically connected to the platform (80), a method of controlling a deployment of the cannula (30), the method comprising:
operating the rotation motor assembly (50) to rotate the cannula (30) to a specific rotational orientation relative to a calibration orientation associated with at least one of the cannula control unit (40) and the platform (80), and
operating the translation motor assembly (60) to translate the cannula control unit (40) to a specific translational position relative to a calibration position associated with at least one of the cannula control unit (40) and the platform (80).
